(19) United States
(12) Patent Application Publication
(10) Pub. No.: US 2012/0309854 A1
(43) Pub. Date: Dec. 6, 2012

(54) ALDOHEXOSE-BASED FLUOROADDITIVES

(75) Inventors: ANILKUMAR RAGHAVANPILLAI, Wilmington, DE (US); Stefan Reinartz, Wilmington, DE (US)

(73) Assignee: E I DU PONT DE NEMOURS AND COMPANY, Wilmington, DE (US)

(21) Appl. No.: 13/470,691

(22) Filed: May 14, 2012

Related U.S. Application Data
(60) Provisional application No. 61/493,648, filed on Jun. 6, 2011.

(51) Int. Cl. C07H 5/06; A01N 25/02; A61K 8/60; C07H 1/00; C09D 5/00; C25D 3/02; C09K 15/20; C09K 13/00; C09K 3/16; B01F 17/16; C11D 3/22; C08K 5/17; C09K 3/00

(52) U.S. Cl. 514/777; 536/18.7; 524/29; 536/55.3; 106/287.26; 106/31.13; 252/182.12; 252/392; 252/79.1; 252/380; 516/12; 516/203; 510/470; 205/317

(57) ABSTRACT

Compositions comprising compounds having an aldohexose moiety, a highly fluorinated moiety, and a nitrogen-containing functional group linking the moieties together are disclosed. The aldohexose-based fluoroadditives are effective in reducing the surface tension of water and are useful in various surfactant applications. Also disclosed are processes for making aldohexose-based fluoroadditives.

### CROSS REFERENCE TO RELATED APPLICATIONS

Subject matter disclosed herein is related to the following copending application titled Aldohexose-Based Fluoroadditives (U.S. PRV 61/493,641) filed contemporaneously herewith, assigned to the assignee of the present invention, and is incorporated by reference herein.

### FIELD OF THE INVENTION

The present invention relates to compositions comprising aldohexose-based fluoroadditives, processes for making thereof, and their use in surfactant applications.

### BACKGROUND OF THE INVENTION

Polyhydroxylated and highly fluorinated compounds have been described as surfactants and repellents. DE 19541788 discloses fluoroalkyl-modified (poly)hydroxy hydrocarbons of formula (1):

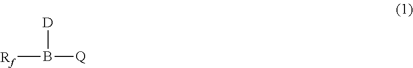

wherein
R_{f}=4-22C perfluoroalkyl or 4-22C monohydroperfluoroalkyl;
B=3-12C alkylene substituted with N and optionally with O or S;
Q=3-18C linear and/or cyclic mono- or poly-hydroxylated hydrocarbon group; and
D=H, or 2-22C alkyl, alkenyl or aryl (all optionally substituted with O, N or S) or 3-22C fluoroalkyl or monohydrofluoroalkyl, (both optionally substituted with O, N or S) or 3-18C linear and/or cyclic mono- or poly-hydroxylated hydrocarbon (optionally substituted with 2-22C alkylene or alkenylene groups which may contain O, N, S, Si).

There remains a need for aldohexose-based fluoroadditives that have improved properties in surfactant applications. One-step processes for making such additives are also desired which provide straightforward product recovery and do not require the use of protective groups or low-temperature manipulations.

### SUMMARY OF THE INVENTION

An aspect of the present invention relates to a composition comprising an aldohexose-based fluoroadditive of formula (I):

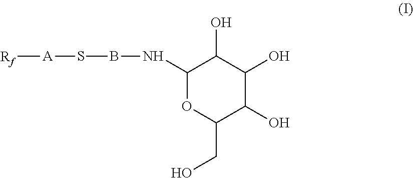

wherein:
R_{f}═C₁-C₁₂ perfluoroalkyl;
A=C₁-C₆ alkylene; and
B═C₁-C₆ alkylene.

Another aspect of the present invention relates to a composition comprising an aldohexose-based fluoroadditive of formula (II):

R¹—CH₂-G₂ (II)

wherein:
R¹═C₁-C₁₂ alkyl or R_{f}-A-G₃-B—,
   R_{f}═C₁-C₁₂ perfluoroalkyl,
   A=C₁-C₆ alkylene,
   B═C₁-C₆ alkylene, and
   G₃=a bond or S;

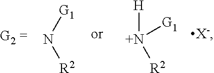

G₁=-CH₂(CHOH)₄CH₂OH or

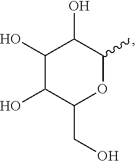

R²═C₁-C₁₂ alkyl or R_{f}-A-G₃-B—, and
X═Cl, Br, or I;

provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then G₂=

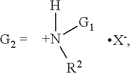

and G₃=a bond; and
when

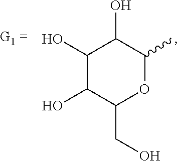

then

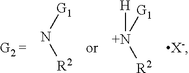

### and G₃=S.

Another aspect of the present invention relates to a process comprising reacting an aldohexose with a compound of formula (III) to yield a compound of formula (I):

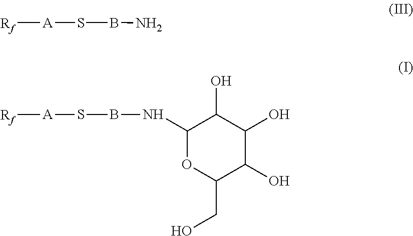

wherein:
R_{f}═C₁-C₁₂ perfluoroalkyl;
A=C₁-C₆ alkylene; and
B═C₁-C₆ alkylene.

Another aspect of the present invention relates to a process comprising:
a) reacting an aldohexose with a compound of formula (IV) to yield a compound of formula (V); and
b) reacting a compound of formula (V) with a compound of formula (VI) to yield a compound of formula (II):

R¹—CH₂—NH₂ (IV)

R¹—CH₂—NH-G₁ (V)

R²—X (VI)

R¹—CH₂-G₂ (II)
wherein:
   R¹═C₁-C₁₂ alkyl or R_{f}-A-G₃-B—,
   R_{f}═C₁-C₁₂ perfluoroalkyl,
   A=C₁-C₆ alkylene,
   B═C₁-C₆ alkylene, and
   G₃=a bond or S;
G₁=-CH₂(CHOH)₄CH₂OH or

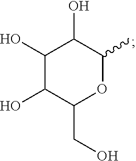

R²═C₁-C₁₂ alkyl or R_{f}-A-G₃-B—;

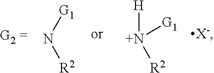

X═Cl, Br, or I;

provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then

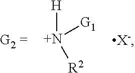

and G₃=a bond; and
when

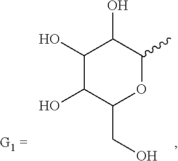

then

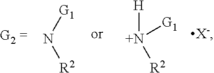

### and G₃=S.

A further aspect of the present invention relates to a method of altering the surface behavior of a medium, comprising adding to the medium an aldohexose-based fluoroadditive selected from the group of compounds of formula (I) and compounds of formula (II).

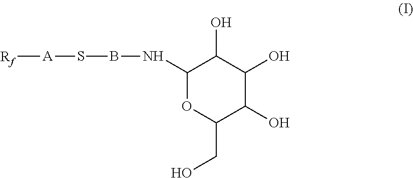

wherein:
R_{f}═C₁-C₁₂ perfluoroalkyl;
A=C₁-C₆ alkylene; and
B═C₁-C₆ alkylene.

R¹—CH₂-G₂ (II)

wherein:
R¹═C₁-C₁₂ alkyl or R_{f}-A-G₃-B—,
   R_{f}═C₁-C₁₂ perfluoroalkyl,
   A=C₁-C₆ alkylene,
   B═C₁-C₆ alkylene, and
   G₃=a bond or S;

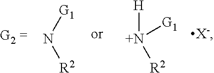

G₁=-CH₂(CHOH)₄CH₂OH or

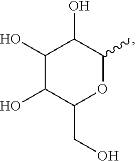

R²═C₁-C₁₂ alkyl or R_{f}-A-G₃-B—, and
X═Cl, Br, or I;

provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then

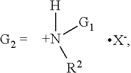

and G₃=a bond; and
when

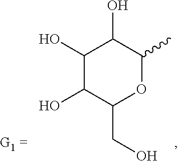

then

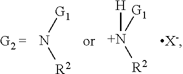

### and G₃=S.

Another aspect of the present invention relates to a process comprising contacting an article with a composition comprising an aldohexose-based fluoroadditive selected from the group of compounds of formula (I) and compounds of formula (II).

### DETAILED DESCRIPTION

As used herein, “alkyl” is meant to include an optionally heteroatom-substituted, linear or branched saturated aliphatic hydrocarbon group having a specified number of carbon atoms. In some embodiments, the alkyl groups described herein contain 1 to 12 carbon atoms and the heteroatom is oxygen.

As used herein, “alkylene” is meant to include an optionally heteroatom-substituted, linear or branched divalent hydrocarbon group having a general formula of CₙH₂ₙ or CₙH₂ₙY. In some embodiments, the alkylene groups contain 1 to 6 carbon atoms (n=1-6) and the heteroatom is oxygen (Y═O).

As used herein, “perfluoroalkyl” is a perfluorinated alkyl group. In some embodiments, the perfluoroalkyl groups herein contain 1 to 12 carbon atoms, preferably 4 to 6 carbon atoms.

As used herein, [symbol] defines the attaching point of a pyranosyl group.

As used herein, “aldohexose” is a monosaccharide with six carbon atoms, having the chemical formula C₆H₁₂O₆, and an aldehyde at position one. The aldohexose has four chiral centers for a total of 16 possible aldohexose stereoisomers.

One aspect of the present invention relates to a composition comprising a compound of formula (I):

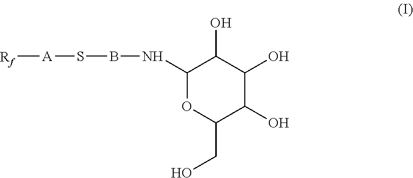

wherein:
R_{f}═C₁-C₁₂ perfluoroalkyl;
A=C₁-C₆ alkylene; and
B═C₁-C₆ alkylene.

Another aspect of the present invention relates to a composition comprising a compound of formula (II):

R¹—CH₂-G₂ (II)

wherein:
R¹═C₁-C₁₂ alkyl or R_{f}-A-G₃-B—,
   R_{f}═C₁-C₁₂ perfluoroalkyl,
   A=C₁-C₆ alkylene,
   B═C₁-C₆ alkylene, and
   G₃=a bond or S;

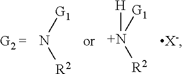

G₁=-CH₂(CHOH)₄CH₂OH or

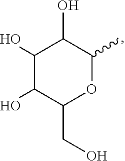

R²═C₁-C₁₂ alkyl or R_{f}-A-G₃-B—, and
X═Cl, Br, or I;

provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then

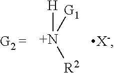

and G₃=a bond; and
when

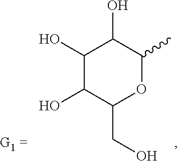

then

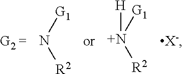

### and G₃=S.

Another aspect of the present invention relates to one-step reductive amination of aldohexose with a compound of formula (III) in the presence of Raney-Ni catalyst to yield a compound of formula (I) as shown in Reaction Scheme 1, wherein R_{f}, A, and B are as defined above.

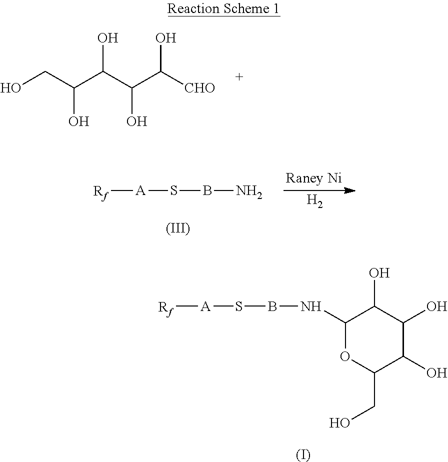

Another aspect of the present invention involves reductive amination of aldohexose with a compound of formula (IV) to yield a compound of formula (V) as shown in Reaction Scheme 2, wherein R¹, and G₁ are as defined above. The secondary amine of formula (V) is further alkylated with an alkyl iodide or a fluoroalkyl iodide of formula (VI) to afford a tertiary amine or an ammounium salt of formula (II) as shown in Reaction Scheme 3, wherein R¹, R², X, G₁, and G₂ are as defined above.

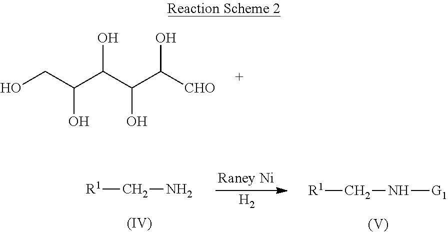

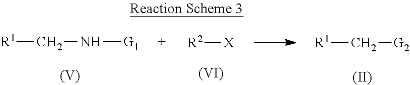

Another aspect of the present invention relates to a method of altering the surface behavior of a medium by adding to the medium an aldohexose-based fluoroadditive selected from the group of compounds of formula (I) and compounds of formula (II). Types of surface behavior that can be altered include wetting, penetration, spreading, leveling, flowing, emulsifying, dispersing, repelling, releasing, lubricating, etching, bonding, and stabilizing. Types of media include coating compositions, lattices, polymers, floor finishes, inks, emulsifying agents, foaming agents, release agents, repellency agents, flow modifiers, film evaporation inhibitors, wetting agents, leveling agents, penetrating agents, cleaners, grinding agents, electroplating agents, corrosion inhibitors, etchant solutions, soldering agents, dispersion aids, antimicrobial agents, pulping aids, rinsing aids, polishing agents, personal care compositions, drying agents, antistatic agents, bonding agents, and mixtures thereof.

Another aspect of the present invention relates to a process comprising contacting an article with a composition comprising an aldohexose-based fluoroadditive selected from the group of compounds of formula (I) and compounds of formula (II). Suitable articles include: polymers, metals, wood, glass, ceramics, bricks, concretes, cements, natural or synthetic stones, tiles, paper, leather, and textile materials. The aldohexose-based fluoroadditives of the present invention can further comprise a medium of the type described above. Suitable polymers include: polycarbonates, polyesters (such as polyethylene terephthalate), polyolefins, polyurethanes, acrylics, polyamides (such as nylon 6, nylon 6,6, and nylon 6,12), polyimides, vinyl polymers (such as polyvinyl chloride), fluoropolymers, silicon polymers (such as polysilanes and polysiloxanes), amino resins, epoxy resins, and phenolic resins. The polymeric articles can be in the form of a fiber, a film, a sheet, a formed or molded part, a laminate, an extruded profile, a coated part, a foamed part, a bead, a particle, or a powder. Typical natural stones include granite and marble, and examples of synthetic stones include solid surface materials such as Corian® from DuPont and quartz surfaces such as Zodiaq® from DuPont.

The aldohexose-based fluoroadditives can be used as surfactants in waxes, finishes, and polishes to improve wetting, leveling, and gloss for floors, furniture, shoe, and automotive care. The aldohexose-based fluoroadditive surfactants of the present invention are useful in a variety of aqueous and non-aqueous cleaning products for glass, tile, marble, ceramic, linoleum and other plastics, metal, stone, laminates, natural and synthetic rubbers, resins, plastics, fibers, and fabrics.

The aldohexose-based fluoroadditive surfactants of the present invention can also be employed as additives in agricultural compositions containing herbicides, weed killers, hormone growth regulators, parasiticides, insecticides, germicides, bactericides, nematocides, microbiocides, defoliants, fertilizers, therapeutic agents, and antimicrobials, with one or more of the following functions: substrate wetting agent, adjuvant, foam inhibitor, dispersant, and emulsion stabilizer. The aldohexose-based fluoroadditive surfactants of the present invention are also suitable as wetting agents for foliage, live stock dips, and live stock skins; as an ingredient in sanitizing, discoloring and cleaning compositions; and in insect repellent compositions.

The aldohexose-based fluoroadditive surfactants of the present invention are suitable for the use in compositions for personal care products (such as shampoos, conditioners, creams, and rinses), cosmetic products for the skin (such as therapeutic or protective creams and lotions, oil and water repellent cosmetic powders, deodorants and anti-perspirants), nail polish, lipstick, toothpastes, fabric care products (such as stain pretreatments and/or stain removers for clothing, carpets and upholstery), laundry detergents, and rinse-aids (for car washes and in automatic dishwashers).

The aldohexose-based fluoroadditive surfactants of the present invention are suitable for the use in the petroleum and gas industries as wetting agents and treatment agents to prevent and remove film evaporation and gas/oil blocking for gas, gasoline, jet fuel, solvents and hydrocarbons.

The aldohexose-based fluoroadditive surfactants of the present invention are further suitable for the use in printing inks, resist inks, developer solutions, photoresists, cleaning solutions, oxide etching compositions, and polishers in the manufacturing, processing, and handling of semiconductors and electronics.

The aldohexose-based fluoroadditive surfactants of the present invention are useful as fire fighting agents in fighting forest fires, dry chemical fire extinguishers, and aerosol-type fire extinguishers.

The aldohexose-based fluoroadditive surfactants of the present invention are further suitable for the use as wetting agents, antifoaming agents, penetrating agents and emulsifying agents in textile and leather industries; lubricants for textiles, nonwoven fabrics and leather treatment; spreading and uniformity agents for fiber finishes; wetting agents for dyeing; binders in nonwoven fabrics; and penetration additives for bleaches.

The aldohexose-based fluoroadditive surfactants of the present invention are further useful as thickening agents in mining industry, metal-working industry, pharmaceutical industry, household, cosmetic and personal products, photography and graphic arts.

The aldohexose-based fluoroadditive surfactants of the present invention can be used as antifogging agents for glass surfaces and photography films, and as antistatic agents for magnetic tapes, phonograph records, floppy disks, disk drives, rubber compositions, PVC, polyester film, photography films, and as surface treatment agents for optical elements (such as glass, plastic, or ceramic beads)

The aldohexose-based fluoroadditive surfactants of the present invention are also useful as foam control agents in polyurethane foams, spray-on oven cleaners, foamed kitchen and bathroom cleansers and disinfectants, aerosol shaving foams, and in textile treatment baths.

The aldohexose-based fluoroadditive surfactants of the present invention are useful as emulsifying agents for polymerization, particularly of fluoromonomers, as latex stabilizers, as mold-release agents for silicones, photoemulsion stabilizers, inorganic particles, and pigments.

### EXAMPLES

### Materials and Methods

All solvents and reagents, unless otherwise indicated, were purchased from Sigma-Aldrich and used directly as supplied. 2-(1H,1H,2H,2H-Perfluorooctylthio)ethylamine and 2-(1H,1H,2H,2H-perfluorohexylthio)ethylamine were prepared by the reaction of 1H,1H,2H,2H-perfluoralkyl iodides with 2-aminoethanethiol, as per the literature procedure (Rondestvedt, C. S., Jr.; Thayer, G. L., Jr. J. Org. Chem. 1977, 42, 2680). 1H,1H,2H,2H,3H,3H,4H,4H-perfluooctyl iodide was prepared by the reaction of perfluorobutyl iodide with ethylene under pressure at elevated temperature in presence of a radical initiator, as described by Brace in U.S. Pat. No. 3,145,222. ¹H and ¹⁹F NMR spectra were recorded on a Brucker DRX 400 or 500 Spectrometer. Chemical shifts are reported in ppm relative to an internal reference (CDCl_{3,} CFCl₃ or TMS).

### Test Method—Measurement of the Critical Micelle Concentration (CMC) and the Surface Tension Beyond CMC

The surface tension measurements of the surfactants were measured in fresh MILLIPORE® filtered water using the Wilhelmy plate method (Acosta, E. J. and Reinartz, S., U.S. Pat. No. 7,385,077) on an automated Krüss tensiometer (Model K11, Krüss USA, Nazareth, Pa.). MILLIPORE® filters are available from Millipore Corporation, Billerica, Mass.

A clean, dry 50 mL plastic beaker was filled with approximately 40 mL of the desired solution for surface tension measurements. The beaker was placed on the sample platform of the Krüss K11 tensiometer. The platinum surface tension probe was removed from the tensiometer hook, rinsed with deionized water and dried with the blue part of the flame from a propane torch. The probe was then air-cooled and reinserted onto the tensiometer hook. The surface tension measurements were performed for surfactant solutions of various dilutions from 1 wt %-0.0001 wt % in water. Ten replicates were tested of each dilution.

The Critical Micelle Concentration (CMC) is defined as the concentration of surfactants above which micelles are spontaneously formed, at which increased concentrations of surfactant essentially no longer reduce the surface tension. The CMC should be as low as possible to provide the lowest cost for effective performance.

To determine CMC, the surface tension was measured as a function of surfactant concentration. Surface tension was then plotted vs. log concentration. The resulting curve had a nearly horizontal portion at concentrations higher than the CMC and had a negative steep slope at concentrations less than the CMC. The CMC is the concentration at which the flat portion and the extrapolated steep slope sections of the curve intersect. The surface tension beyond CMC was the value in the flat portion of the curve.

### Example 1

### N-2-(6,6,7,7,8,8,9,9,10,10,11,11,12,12,12-tridecafluorooctylthio)ethyl-D-glucopyranosylamine, 1

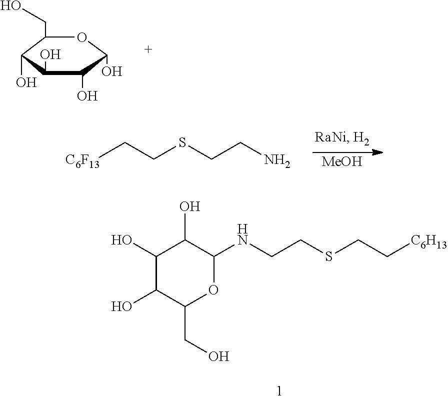

This example illustrates the synthesis of Compound 1, an example of a compound of formula (I).

A 200 mL shaker tube containing a mixture of 2-(1H,1H,2H,2H-perfluorooctylthio)ethylamine (5.8 g, 0.0137 mol), D-glucose (2.25 g, 0.0125 mol), Raney-Ni catalyst (1.5 g slurry, ˜0.75 g catalyst), and methanol (40 mL) was agitated at 60° C. for 12 h under an H₂ atmosphere (160 psi). The catalyst was then removed by filtration of the reaction mixture through Celite. The filtrate was concentrated under vacuum to obtain 6.2 g (82% yield) of a white solid. The crude product was recrystallized from diethyl ether/ethanol (10:1) to obtain 1.7 g of a pale yellow solid 1: LC-MS (API-ES+), major peak M+H=586; ¹⁹F NMR (DMSO-d6): δ −81.3 (m, 3F), −113.8 (m, 2F), −122.3 (m, 2F), −123.2 (m, 2F), −123.4 (2F), −126.4 (m, 2F); ¹³C NMR (DMSO-d6) δ 126 MHz, δ (for non-fluorinated carbons): 91.03, 78.0, 74.3, 73.6, 71.2, 61.6, 45.9, 32.0, 31.4 (t, ²J_{CF}=68.8 Hz), 20.1.

### Example 2

### N-2-(6,6,7,7,8,8,9,9,9-nonafluorohexylthio)ethyl-D-glucopyranosylamine, 2

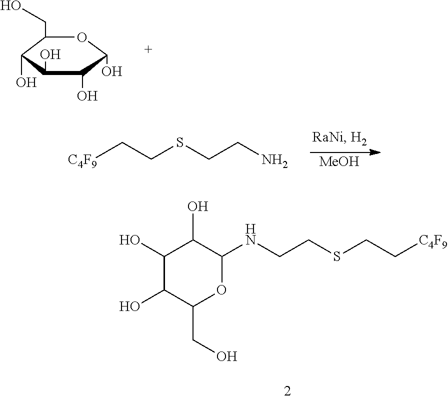

This example illustrates the synthesis of Compound 2, another example of a compound of formula (I).

The procedure described in Example 1 was followed using 2-(1H,1H,2H,2H-perfluorohexylthio)ethylamine (4.44 g, 0.01375 mol) and D-glucose (2.25 g, 0.0125 mol). A crude product was isolated (5.4 g, 88% yield) as a white solid. The crude product was recrystallized from diethyl ether/ethanol (10:1) to obtain 1.7 g of pale yellow solid 2: ¹⁹F NMR (CD₃OD): δ −81.5 (m, 3F), −113.9 (m, 2F), −124.6 (m, 2F), −126.3 (m, 2F); ¹³C NMR (DMSO-d6) δ 126 MHz, (for non-fluorinated carbons): 91.7, 78.3, 74.2, 73.8, 71.4, 62.1, 46.6, 32.8, 31.7 (t, ²J_{CF}=68.0 Hz), 20.9.

### Example 3

### 1-Deoxy-1-[(5,5,6,6,7,7,8,8,8-nonafluorooctylhexylammonium]-hexitol iodide, 3

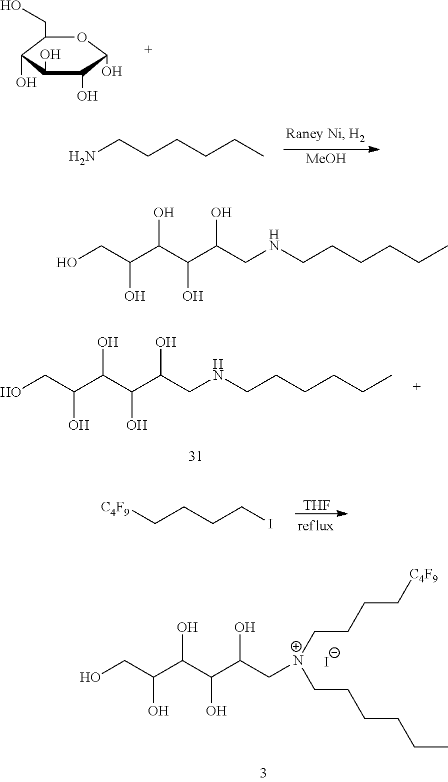

This example illustrates the synthesis of Compound 3, an example of a compound of formula (II).

The procedure described in Example 1 was followed using hexyl amine (7.6 g, 0.075 mol) and D-glucose (13.5 g, 0.075 mol) to provide 6.7 g (33% yield) of 1-deoxy-1-hexylamino-hexitol (3l) as an off-white solid after recrystallization from hot EtOH: LC/MS (API-ES+) M+H=266.

1-Deoxy-1-hexylamino-hexitol (3l) (2.5 g, 0.0094 mol), dry THF (50 mL) and 1H,1H,2H,2H,3H,3H,4H,4H-perfluorooctyl iodide (4 g, 0.0099 mol) were added under nitrogen to a three-neck 100 mL round bottom flask fitted with stir bar, condenser, and thermocouple. The mixture was allowed to reflux for 2 days and then cooled to room temperature. The precipitate was filtered off (mainly starting material (3l) determined by LC/MS), and the filtrate was concentrated under vacuum to obtain a yellow oil 3 (4.4 g, 70% yield): LC/MS (API-ES+) M+H=512; ¹H NMR (CDCl₃) δ: 0.9 (t, 6.8 Hz, 3H), 1.25-1.32 (m, 8H), 1.70-1.78 (m, 4H), 2.12 (m, 2H), 2.85-2.92 (m, 6H), 3.31 (m, 4H), 3.62-3.80 (m, 2H), 4.32 (bs, 5H).

### Example 4

This example illustrates the surface tension measurements of Compounds 1-3 of the invention and comparative examples.

Comparative Example A was a commercially available nonionic surfactant from E. I. du Pont de Nemours and Company, Wilmington, Del. containing a mixture of ethoxylated perfluoroalkylethanol ranging from 2-16 carbon atoms, predominantly 8 carbon atoms, in ethylene glycol and water. The level of ethoxylation is ˜10-11 ethylene oxide (EO) units.

Comparative Example B was a commercially available nonionic surfactant from E. I. du Pont de Nemours and Company, Wilmington, Del. containing a mixture of ethoxylated perfluoroalkylethanol ranging from 2-16 carbon atoms, predominantly 6, 8 and 10 carbon atoms, in ethylene glycol and water. The level of ethoxylation is higher than that of Comparative Example A (˜19-20 EO units).

The surface tensions of the aldohexose-based fluoroadditive surfactants (1-3) in water were measured as described above in the Test Method. The critical micelle concentration (CMC) and the surface tension beyond CMC of the aldohexose-based fluoroadditives (1-3) were compared with those of Comparative Examples A and B, which contain longer fluoroalkyl chains. The results are summarized in Table 1.

TABLE 1
CMC and Surface Tension beyond CMC

| | Critical Micelle Concn. | Surface Tension |
|---|---|---|
| Compound | (% by weight) | Beyond CMC (mN/m) |
| 1 | 0.0085 | 18.6 |
| 2 | 0.00675 | 18.9 |
| 3 | 0.0375 | 22.5 |
| Comparative Ex. A | 0.015 | 21.1 |
| Comparative Ex. B | 0.0925 | 24.5 |

All compounds were very effective in reducing the surface tension of water from 72 mN/m (pure water) to ˜25 mN/m or lower at concentrations below 0.05 wt %. Compounds 1 and 2 showed better (lower) critical micelle concentrations and surface tensions than Comparative Examples A and B. Compound 3 showed better (lower) critical micelle concentration and surface tension than Comparative Example B.

What is claimed is:

1. A composition comprising a compound of formula (II):
R¹—CH₂-G₂ (II)
wherein:
R¹═C₁-C₁₂ alkyl or R_{f}-A-G₃-B—,
R_{f}═C₁-C₁₂ perfluoroalkyl,
A=C₁-C₆ alkylene,
B═C₁-C₆ alkylene, and
G₃=a bond or S; 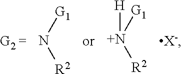
G₁=-CH₂(CHOH)₄CH₂OH or 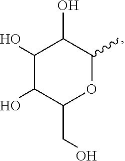
R²═C₁-C₁₂ alkyl or R_{f}-A-G₃-B—, and
X═Cl, Br, or I;
provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then 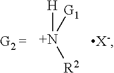
and G₃=a bond; and
when 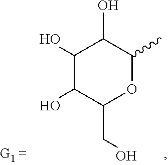
then 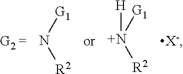
and G₃=S.

2. A process comprising:
a) reacting an aldohexose with a compound of formula (IV) to yield a compound of formula (V); and
b) reacting a compound of formula (V) with a compound of formula (VI) to yield a compound of formula (II):
R¹—CH₂—NH₂ (IV)
R¹—CH₂—NH-G₁ (V)
R²—X (VI)
R¹—CH₂-G₂ (II)
wherein:
R¹═C₁-C₁₂ alkyl or R_{f}-A-G₃-B—,
R_{f}═C₁-C₁₂ pertluoroalkyl,
A=C₁-C₆ alkylene,
B═C₁-C₆ alkylene, and
G₃=a bond or S;
G₁=-CH₂(CHOH)₄CH₂OH or 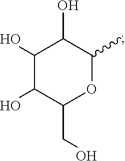
R²═C₁-C₁₂ alkyl or R_{f}-A-G₃-B—;
X═Cl, Br, or I; 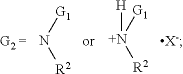
provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then 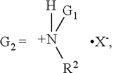
and G₃=a bond; and
when 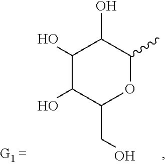
then 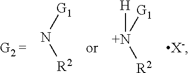
and G₃=S.

3. A method of altering the surface behavior of a medium, comprising adding to the medium an aldohexose-based fluoroadditive selected from the group of compounds of formula (II):
R¹—CH₂-G₂ (II)
wherein:
R¹═C₁-C₁₂ alkyl or R_{f}-A-G₃-B—,
R_{f}═C₁-C₁₂ pertluoroalkyl,
A=C₁-C₆ alkylene,
B═C₁-C₆ alkylene, and
G₃=a bond or S; 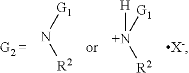
G₁=-CH₂(CHOH)₄CH₂OH or 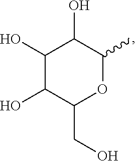
R²═C₁-C₁₂ alkyl or R_{f}-A-G₃-B—, and
X═Cl, Br, or I;
provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then 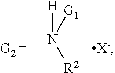
and G₃=a bond; and
when 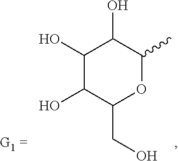
then 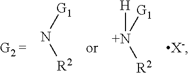
and G₃=S.

4. The method of claim 3, wherein adding the aldohexose-based fluoroadditive to the medium lowers the surface tension of the medium.

5. The method of claim 4, wherein the surface behavior is selected from the group consisting of wetting, penetration, spreading, leveling, flowing, emulsifying, dispersing, repelling, releasing, lubricating, etching, bonding, and stabilizing.

6. The method of claim 5, wherein the medium is selected from the group consisting of: coating compositions, lattices, polymers, floor finishes, inks, emulsifying agents, foaming agents, release agents, repellency agents, flow modifiers, film evaporation inhibitors, wetting agents, leveling agents, penetrating agents, cleaners, grinding agents, electroplating agents, corrosion inhibitors, etchant solutions, soldering agents, dispersion aids, antimicrobial agents, pulping aids, rinsing aids, polishing agents, personal care compositions, drying agents, antistatic agents, bonding agents, and mixtures thereof.

7. A process comprising contacting an article with a composition comprising an aldohexose-based fluoroadditive selected from the group of compounds of formula (II):
R¹—CH₂-G₂ (II)
wherein:
R¹═C₁-C₁₂ alkyl or R_{f}-A-G₃-B—,
R_{f}═C₁-C₁₂ perfluoroalkyl,
A=C₁-C₆ alkylene,
B═C₁-C₆ alkylene, and
G₃=a bond or S; 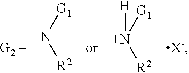
G₁=-CH₂(CHOH)₄CH₂OH or 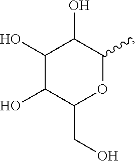
R²═C₁-C₁₂ alkyl or R_{f}-A-G₃-B—, and
X═Cl, Br, or I;
provided that:
when R¹═C₁-C₁₂ alkyl, then R²═R_{f}-A-G₃-B—;
when R¹═R_{f}-A-G₃-B—, then R²═C₁-C₁₂ alkyl;
when G₁=-CH₂(CHOH)₄CH₂OH, then 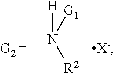
and G₃=a bond; and
when 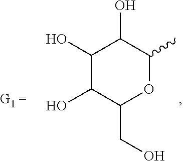
then 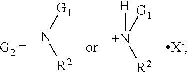
and G₃=S.

8. The process of claim 7, wherein the composition further comprises a medium.

9. The process of claim 8, wherein the medium is selected from the group consisting of: coating compositions, lattices, polymers, floor finishes, inks, emulsifying agents, foaming agents, release agents, repellency agents, flow modifiers, film evaporation inhibitors, wetting agents, leveling agents, penetrating agents, cleaners, grinding agents, electroplating agents, corrosion inhibitors, etchant solutions, soldering agents, dispersion aids, antimicrobial agents, pulping aids, rinsing aids, polishing agents, personal care compositions, drying agents, antistatic agents, bonding agents, and mixtures thereof.

10. The process of claim 7, wherein the article comprises a material selected from the group consisting of polymers, metals, wood, glass, ceramics, bricks, concretes, cements, natural or synthetic stones, tiles, paper, leather, and textile materials.

11. The process of claim 10, wherein the polymeric article is in the form of a fiber, a film, a sheet, a formed or molded part, a laminate, an extruded profile, a coated part, a foamed part, a bead, a particle, or a powder.

12. An article produced by the process of claim 7.
